(19) United States
(12) Patent Application Publication
(10) Pub. No.: US 2025/0262188 A1
(43) Pub. Date: Aug. 21, 2025

(54) Methods of Increasing the Plasma Drug Exposure of Anticancer Agents

(71) Applicant: Scandion Oncology A/S, Copenhagen Ø (DK)

(72) Inventors: Peter Michael Vestlev, Copenhagen Ø (DK); Nicklas Lindland Roest, Copenhagen Ø (DK)

(21) Appl. No.: 18/569,260

(22) PCT Filed: Jun. 14, 2022
(86) PCT No.: PCT/EP2022/066097

§ 371 Date: Dec. 12, 2023

(30) Foreign Application Priority Data
Jun. 14, 2021 (EP) 21179186.8
Jun. 14, 2021 (EP) 21179188.4
Sep. 6, 2021 (EP) 21195165.2
Sep. 27, 2021 (EP) 21199204.5
Jan. 10, 2022 (EP) 22150817.9
Mar. 6, 2022 (EP) 22160364.0

(51) Int. Cl. A61K 31/41; A61K 31/4745

(52) U.S. Cl. CPC A61K 31/41; A61K 31/4745

(57) ABSTRACT

The present invention relates to treatment regimens for increasing efficacy and potentecy of chemotherapeutic agents in the treatment of cancers, in particular to methods increasing the plasma drug exposure (AUC) and/or plasma half-lifes of an anti-cancer agent by administering an effective amount of a UGT1A1 and/or ABCG2 inhibitor.

### CROSS REFERENCE TO RELATED APPLICATIONS

The present application is a U.S. national phase of International Application No. PCT/EP2022/066097, filed Jun. 14, 2022, which claims priority to European Patent Application Nos. 21179186.8, filed Jun. 14, 2021, 21179188.4, filed Jun. 14, 2021, 21195165.2, filed Sep. 6, 2021, 21199204.5, filed Sep. 27, 2021, 22150817.9, filed Jan. 10, 2022, 22160634.0, filed Mar. 6, 2022, all of which are incorporated herein by reference in their entirety.

### FIELD OF THE INVENTION

The present invention relates to treatment regimens for increasing efficacy and potentecy of chemotherapeutic agents in the treatment of cancers, in particular to methods increasing the plasma drug exposure (AUC) and/or plasma half-lifes of an anti-cancer agent by administering an effective amount of a UGT1A1 and/or ABCG2 inhibitor.

### BACKGROUND OF THE INVENTION

Cancer is an overwhelming burden to our society with approximately 14 million new cases of cancer diagnosed in 2014. Despite the introduction of many new treatment modalities/options, de novo or acquired resistance to the applied treatments still represents the major cause of death from cancer.

For example colorectal cancer (CRC) is one of the most common cancers in Europe and worldwide over 1.8 million new cases and 881,000 deaths are estimated to occur in 2018. Unfortunately, a large proportion of these patients will develop metastatic disease (mCRC) despite prior adjuvant treatment and approximately 20% of newly diagnosed CRC patients did present with metastatic disease before the introduction of screening.

The standard of care to patients with local recurrence or mCRC is either surgery and/or chemotherapy and targeted therapy with monoclonal antibodies. For incurable patients, standard drugs are 5-flourouracil and derivatives, oxaliplatin, irinotecan, bevacizumab and panitumumab or cetuximab. The anti-cancer agent irinotecan is most often prescribed in combination with 5-flourouracil and leucovorin (FOLFIRI).

One major problem in the treatment of many cancers, including mCRC, is the frequent development of drug resistance. In practical terms, this means the cancer continues to either grow during anti-cancer treatment (de novo resistance) or re-grow after an initial response to anti-cancer treatment (acquired resistance). Acquired resistance is often accompanied by cross-resistance to other anti-cancer drugs.

Every year approximately 1000 Danish patients are diagnosed with Pancreatic Cancer (PC). Among the few approved treatments for inoperable PC is FOLFIRINOX which is a combination of irinotecan, 5-flourouracil and leucovorin in combination with oxaliplatin, or the combination of gemcitabine and nab-paclitaxel that was introduced in 2013 as a result of a large phase III study. Pancreatic cancer patients are fragile and only about 50% of the patients tolerate the treatments named above, the rest of the patients are treated with gemcitabine as monotherapy or best supportive care.

Pancreatic cancer has a high frequency of primary (de novo) resistance against chemotherapy, but also fast development of secondary (acquired) resistance is a great problem as most patients, who have an initial positive effect of chemotherapy, will experience disease progression at a later time-point. In both pancreatic cancer and metastatic colorectal cancer, which both are cancers with few treatment options, irinotecan is an important anti-cancer agent in the palliative phase of the illness.

Irinotecan is often used in the treatment of mCRC and pancreatic cancer. However, most of the patients rapidly develop resistance to this drug. Upregulation of the ABCG2 drug efflux pump is often observed in irinotecan resistant disease and since the active metabolite of irinotecan, SN-38, is a substrate for this efflux pump, ABCG2 upregulation is considered a major resistance mechanism against irinotecan treatment.

Irinotecan is an intravenously administered medicinal product, and the overall pharmacokinetic exposure is therefore determined by the rate of key metabolic pathways for its active metabolite SN-38. Usually, administration of Irinotecan results in a rapid increase in the concentration of irinotecan, followed by a comparably rapid conversion to the active substance SN-38 by carboxylestarases. In the liver, SN-38 is subsequently glucoronidated by UGT1A1 to form the inactive and more water-soluble SN-38-glucoronide conjugate which can be excreted from hepatic cells via e.g. ABCG2 to be eliminated from the body via biliary excretion to the feces.

Using standard intravenous formulations of Irinotecan or SN-38, an increased exposure is only possible with increased dosage of irinotecan, however as higher doses are linked to significant toxicity, for most patients, this is not a feasible option. Efforts have been pursued to increase the exposure of Irinotecan and SN-38 by extending the half-life of Irinotecan (parent compound) through liposome-formulation (e.g. Onivyde) resulting in somewhat decreased clearance of irinotecan from the blood stream and thereby a limited increase in the time, which Irinotecan circulates in the body and by a corresponding extension the AUC of both Irinotecan and SN-38. Although this approach has to some extent been shown to result in a sustained exposure for some prolonged period of time, significant issues persist regarding tolerability and maintain therapeutically effective plasma concentration of SN-38 during treatment of cancers, in particular resistant cancers.

The compound SCO-101, also known as NS3728 was first described in WO 2000/24707. SCO-101 has later been shown to be an effective potentiator of a range of anti-cancer agents and is currently being developed for cancer combination therapies, in particular for treatment of resistant cancers. WO 2017/198700 describes SCO-101 and its use in combination therapies for treatment of cancers.

Accordingly, the present invention provides improvements offering solutions to drawbacks of this background art.

### SUMMARY OF THE INVENTION

The present inventors have found that SCO-101 is a potent inhibitor of UGT1A1 and ABCG2 which the present inventors have found to surprisingly influence, inter alia, the metabolism and clearance of anti-cancer agents from the human body. Data from a study performed by the present inventors unexpectedly found that by administering a regulator compound such as SCO-101 the pharmacokinetic exposure as defined by the Area Under the Curve (AUC) of an anti-cancer agent such as irinotecan and/or its active metabolite SN-38 can be manipulated by increasing the Cₘₐₓ and increasing the half-life (t½) of the anti-cancer agent through a reduced metabolism and clearance from the human body of the anti-cancer agent. This manipulation enables a specific and intentional increase in the pharmacokinetic exposure (AUC) of the anti-cancer agent, which is increased compared to the pharmacokinetic exposure (AUC) resulting from conventional drug formulations with the anti-cancer agent. Even more unexpected, it was found that the increased pharmacokinetic exposure (AUC) and/or Cₘₐₓ and/or half-life (t½) of the anti-cancer agent could be achieved with a reduced dose of the anti-cancer agent compared to administration of conventional doses. These findings support a favorable tolerability profile and demonstrates the superiority of the present invention over conventional anti-cancer agent formulations.

Accordingly, in a first aspect the present invention provides a method of increasing the plasma drug exposure (AUC) of an anti-cancer agent comprising administering to a patient, receiving the anti-cancer agent for treatment of a cancer, a regulator compound effectively inhibiting the degradation, clearance, and/or binding of the anti-cancer agent and/or a therapeutically active metabolite thereof, and thereby increasing the plasma drug exposure of an anti-cancer agent.

In a further aspect, the invention provides a combination drug comprising, separately or together,
a) an anti-cancer agent, and
b) a regulator compound,
   wherein the amount of the regulator compound effectively increases the plasma drug exposure (AUC) of the anti-cancer agent when administering the combination drug to a patient.

In a further aspect, a combination drug is provided comprising, separately or together,
a) an anti-cancer agent; and
b) a regulator compound;
   for use in the treatment of cancer in a patient, wherein an amount of the regulator compound effectively increases the plasma drug exposure (AUC) of the anti-cancer agent when administering the combination drug to a patient.

In a further aspect, a regulator compound is provided for use in increasing the plasma drug exposure (AUC) of an anti-cancer agent in a patient receiving the anti-cancer agent for treatment of a cancer, wherein the regulator compound is an inhibitor of the degradation, clearance, and/or binding of the anti-cancer agent and/or a therapeutically active metabolite thereof, and thereby increasing the plasma drug exposure of an anti-cancer agent.

### DESCRIPTION OF DRAWINGS AND FIGURES

The figures included herein are illustrative and may be simplified for clarity, and they may merely show details which are essential to the understanding of the invention, while other details may have been left out. In the figures and drawing included herein:

FIG. 1 shows the plasma concentrations of anti-cancer agent SN-38 and/or regulator compound SCO-101 for different dosing experiments with these compounds.

FIG. 2 shows the correlation between SCO-101 and SN-38 plasma concentrations (AUC) in patients.

FIG. 3 shows the uptake of SN-38 in cell monolayers of both the SN-38 resistant (FIG. 3A) and the parental (FIG. 3B) HT29 cell lines. FIG. 3C correspond to FIG. 3A, and FIG. 3D to FIG. 3B where the amount of SN-38 in cell monolayers have been normalized with respect to the control to provide the fold-change resulting from combination treatment with SCO-101.

FIG. 4 shows the effect of increasing concentrations of SCO-101 on the uptake of SN-38 in monolayers of the HT29 SN-38 resistant (FIG. 4A) and parental (FIG. 4B) cell lines.

FIG. 5 shows the effect of four kinase inhibitors (Sphinx31, Srpin340, Tomivorsetib and CGP57380), 25 μM SCO-101 and 1 μM KO143 on the uptake of SN-38 in monolayers of of the HT29 SN-38 resistant (FIG. 5A) and parental (FIG. 5B) cell lines.

### INCORPORATION BY REFERENCE

All publications, patents, and patent applications referred to herein are incorporated by reference to the same extent as if each individual publication, patent, or patent application was specifically and individually indicated to be incorporated by reference. In the event of a conflict between a term herein and a term in an incorporated reference, the term herein prevails.

### DETAILED DESCRIPTION OF THE INVENTION

The features and advantages of the present invention are readily apparent to a person skilled in the art by the below detailed description of embodiments and examples of the invention with reference to the figures and drawings included herein.

### Definitions

The term “irinotecan” as used herein refers to a topoisomerase inhibitor of the general formula.

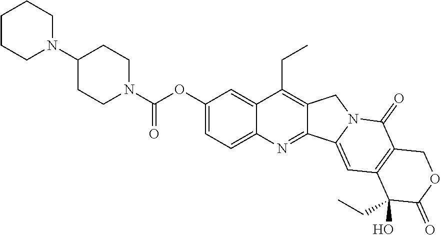

### Irinotecan is Also Known Under the Trade Name Camptosar

The term “SN-38” as used herein refers to a topoisomerase I inhibitor of the general formula

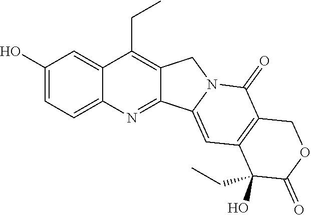

SN-38 is the active metabolite of irinotecan.

The term “SCO-101” or “NS3728” refers to a compound of the general formula:

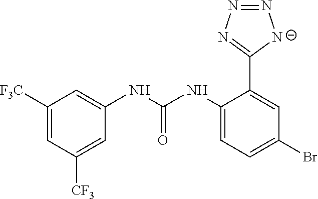

or a pharmaceutically acceptable salt thereof.

The term “pharmacokinetic exposure” or “Area Under the Curve (AUC)” as used herein interchangeably is a term well known in the art and refers to the definite integral of a curve plotting the blood plasma concentratiom of an administered drug as a function of time.

The term “Cₘₐₓ” as used herein is a term well known in the art and refers to the peak concentration that a drug achieves in a specified compartment, such as in the blood plasma, after the drug has been administrated and before administration of any subsequent doses of the drug.

The term “half-life” or “t_{1/2},” as used herein interchangeably is a term well known in the art and refers to the time it takes for the concentration of an administered drug in a specified compartment, such as in the blood plasma, to be reduced by half (50%). Usually the initial half-life is calculated as the time it takes to reduce the concentration of the drug to half of the maximum plasma concentration (Cₘₐₓ).

The term “anti-cancer agent” as used herein refers to a chemotherapeutic agent, that has activity against a susceptible cancer cell.

The term “UGT1A1 inhibitor” as used herein refers to a molecule which is capable of binding to human uridine diphosphate (UDP)-glycuronosyl transferase (UGT1A1), particularly in humans or animals and decrease the enzymatic activity of the UGT1A1. UGT1A1, for example found in the liver of humans, gluronidation a range of different molecules hereinunder several anti-cancer agents, bilirubin, and other molecules usually converting them to less active and more water-soluble forms. Whether or not a particular molecule is an UGT1A1 inhibitor can be determined by examining the molecule's IC50 in vitro in a UGT1A1 inhibition assays known in the art.

The term “ABCG2 inhibitor” as used herein refers to a molecule which is capable of binding to the ABCG2 efflux pump in human or animal cells and thereby reduce it pumping capacity. Efflux pumps are proteinaceous transporters localized in the cytoplasmic membrane of all kinds of cells. They are active transporters, meaning that they require a source of chemical energy to perform their function.

The term “UGT1A1 substrate” as used herein refers to a molecule which is capable of being chemically transformed by enzymatic aid of uridine diphosphate (UDP)-glycuronosyl transferase (UGT1A1). Typically, a UGT1A1 substrate is capable of being glucuronidated by UGT1A1.

The term “recommended dose” as used herein refers to the recommended dosage or dose of an anti-cancer agent as approved by a medicines agency, such as the EMA, the FDA or the Danish Medicines Agency. The doses of the anti-cancer agent irinotecan (CAMPTOSAR) recommended by the FDA are:
Colorectal cancer combination regimen 1: Irinotecan 125 mg/m2; intravenous infusion over 90 minutes on days 1, 8, 15, 22 with Leucovorin 20 mg/m2 intravenous bolus infusion on days 1, 8, 15, 22 followed by 5-Fluorouracil intravenous bolus infusion on days 1, 8, 15, 22 every 6 weeks.
Colorectal cancer combination regimen 2: Irinotecan 180 mg/m2 intravenous infusion over 90 minutes on days 1, 15, 29 with LV, 200 mg/m2 intravenous infusion over 2 hours on days 1, 2, 15, 16, 29, 30 followed by 5-FU 400 mg/m2 intravenous bolus infusion on days 1, 2, 15, 16, 29, 30 and 5-FU 600 mg/m2 intravenous infusion over 22 hours on days 1, 2, 15, 16, 29, 30.
Colorectal cancer single agent regimen 1: Irinotecan 125 mg/m2 intravenous infusion over 90 minutes on days 1, 8, 15, 22 then 2-week rest. (2.2).
Colorectal cancer single agent regimen 2: Irinotecan 350 mg/m2 intravenous infusion over 90 minutes on day 1 every 3 weeks. (2.2)
   Based on medicines agencies' recommendations as described above, it is known to the skilled person, which dose of an anti-cancer agent is the recommended dose.

Body surface area (BSA) is commonly used in the calculation of drug dosages and the amounts of fluids to be administered IV in the medical setting. The body surface area is expressed in m², and a dose in milligram (mg) can thus be normalized according to body surface area, i.e. expressed as mg/m². For many clinical purposes, BSA is a better indicator of metabolic mass than body weight because it is less affected by abnormal adipose mass. Doses of chemotherapeutic agents are often provided in mg/m². The typical body surface area is generally taken to be 1.7 m², but the body surface area depends on more than just height and weight. Other influential factors include the age and gender of the individual. There was an average BSA of 1.73 m² for 3,000 cancer patients from 1990 to 1998 in a European Organisation for Research and Treatment of Cancer (EORTC) database.

During 2005 there was an average BSA of 1.79 m2 for 3,613 adult cancer patients in the UK. Among them the average BSA for men was 1.91 m² and for women was 1.71 m². BSA is a long time term/unit generally practiced and recognized by doctors/oncologists world wide.

The term “pharmaceutically acceptable salt” as used herein refers, without limitation, to inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Such salts may be formed by procedures well known and described in the art.

The term “FOLFIRI” used herein refers to a chemotherapy regimen for treatment of cancer, such as colorectal cancer. It is made up of the following drugs FOL-folinic acid (leucovorin), a vitamin B derivative used as a “rescue” drug for high doses of the drug methotrexate but increases the cytotoxicity of 5-fluorouracil; F—fluorouracil (5-FU), a pyrimidine analog and antimetabolite which incorporates into the DNA molecule and stops synthesis; and IRI—irinotecan (Camptosar), a topoisomerase inhibitor, which prevents DNA from uncoiling and duplicating. One recommended dosage regimen consists of: irinotecan (180 mg/m² IV over 90 minutes) concurrently with folinic acid (400 mg/m² [or 2×250 mg/m²] IV over 120 minutes) followed by 5-fluorouracil (400-500 mg/m² IV bolus) then 5-fluorouracil (2400-3000 mg/m² intravenous infusion over 46 hours).

The term “treatment regimen” used herein refers to a method for treating cancer cells and/or tumours comprising cancer cells in a patient that includes administering simultaneously or sequentially therapeutically effective amounts of the regulator compound and the anti-cancer agent(s) of the invention.

The phrase “therapeutically effective amount” as used herein refers to that amount of a molecule, composition, kit or treatment regimen as a whole that produces some desired local or systemic effect, typically at a reasonable benefit/risk ratio in the context of a treatment regimen or method. The therapeutically effective amount of such substance will vary depending upon the patient and disease condition being treated, the weight and age of the patient, the severity of the disease condition, the manner of administration and the like. For example, certain compositions described herein may be administered in a sufficient amount to produce a desired effect at a reasonable benefit/risk ratio applicable to such treatment.

The terms “substantially” or “approximately” or “about”, as used herein refers to a reasonable deviation around a value or parameter such that the value or parameter is not significantly changed.

These terms of deviation from a value should be construed as including a deviation of the value where the deviation would not negate the meaning of the value deviated from. For example, in relation to a reference numerical value the terms of degree can include a range of values plus or minus 10% from that value. For example, deviation from a value can include a specified value plus or minus a certain percentage from that value, such as plus or minus 9%, 8%, 7%, 6%, 5%, 4%, 3%, 2%, or 1% from the specified value.

The term “and/or” as used herein is intended to represent an inclusive “or”. The wording X and/or Y is meant to mean both X or Y and X and Y. Further the wording X, Y and/or Z is intended to mean X, Y and Z alone or any combination of X, Y, and Z.

The terms “comprise” and “include” as used throughout the specification and the accompanying items as well as variations such as “comprises”, “comprising”, “includes” and “including” are to be interpreted inclusively. These words are intended to convey the possible inclusion of other elements or integers not specifically recited, where the context allows.

The articles “a” and “an” are used herein refers to one or to more than one (i.e., to one or at least one) of the grammatical object of the article. By way of example, “an element” may mean one element or more than one element.

Terms like “preferably”, “commonly”, “particularly”, and “typically” are not utilized herein to limit the scope of the invention or, unless specifically described otherwise, to imply that certain features are critical, essential, or even important to the structure or function of the invention. Rather, unless specifically described otherwise, these terms are merely intended to highlight alternative or additional features that can or cannot be utilized in a particular embodiment of the present invention.

The term “de novo resistance” as used herein about cancer cells refers to cancer cells not sensitive to chemotherapy, without any prior exposure to the chemotherapy.

The term “acquired resistance” as used herein about cancer cells refers to cancer cells that have shown prior sensitivity to a chemotherapy and that have developed reduced sensitivity to the chemotherapy by after treating it with the chemotherapy.

The term “re-sensitizing” as used herein about cancer cells refers to cancer cells that have shown resistance to chemotherapy-either de novo resistance or acquired resistance, and which cells after an intervention—for example treatment with a drug that reverses sensitivity-regain or develop sensitivity to the chemotherapy drug.

The invention provides for a method of increasing the plasma drug exposure (AUC) of anti-cancer agent(s) comprising administering to a patient, receiving the anti-cancer agent(s) for treatment of a cancer, a regulator compound effectively inhibiting the degradation, clearance, and/or binding of the anti-cancer agent and/or a therapeutically active metabolite thereof, and thereby increasing the plasma drug exposure of an anti-cancer agent.

In some embodiments of the invention the administration of the regulator compound also increases the plasma half-life (t_{1/2}) of the anti-cancer agent, due to the regulator compounds degradation, clearance, and/or binding of the anti-cancer agent. The body may degrade the anti-cancer agent through metabolic processes into an inactive or less active metabolite or it may clear the anti-cancer agent modified or unmodified through renal clearance, or the anti-cancer agent may be bound by another compound, thereby masking the anti-cancer agent from being active against a cancer, or a combination of the foregoing. For example, irinotecan undergoes in the body metabolic first conversion into its active metabolite SN-38 by carboxylesterases and subsequently conversion by UGT1A1 glucuronidation into the inactive and more water-soluble SN-38-glucoronide, which is excreted from hepatic cells, e.g. via the ABCG2 efflux pump, and eliminated from the body via biliary excretion to the feces.

In particular embodiments the method of the invention the plasma drug exposure (AUC) and/or the plasma half-life (t_{1/2}) is increased by more that 10%, such as more than 25%, such as more than 30%, such as more than 40%, such as more than 50%, such as more than 60%, such as more than 70%, such as more than 75%, such as more than 80%, such as more than 90%, such as more than 100%, such more than 120%, such as more than 140%, such as more than 160%, such as more than 180%, such as more than 200%, such as more than 220%, such as more than 240%, such as more than 260%, such as more than 280%, such as more than 300%, as compared to if the anti-cancer agent was administered without also administering the regulator compound.

The administration of the anti-cancer agent provides for a maximum plasma concentration (Cₘₐₓ) of the anti-cancer agent. In some embodiments of the invention the administration of the regulator compound provides for an increased Cₘₐₓ of the anti-cancer agent compared to the Cₘₐₓ when administering the anti-cancer agent without the regulator compound, optionally even compared to the Cₘₐₓ when using a higher dose of the anti-cancer agent.

In some embodiments of the invention the plasma drug exposure (AUC) of the administered anti-cancer agent when also administering the regulator compound is increased, even compared to the AUC of the anti-cancer agent being administered at a higher dose without the regulator compound.

### Regulator Compounds

The regulator compound is preferably a UGT1A1 and/or ABCG2 inhibitor. The UGT1A1 and/or ABCG2 inhibitor of the invention preferably has an IC50 of 20 μM or less, such as 15 μM or less, such as 10 μM or less, such as 5 μM or less. In one embodiment, the IC50 is 5 μM or less, such as 4 μM or less, such as 3 μM or less, such as 2 μM or less, for example between 0.1 μM and 2.0 μM. In some embodiments, the UGT1A1 and/or ABCG2 inhibitor is non-competitive, while in other embodiments the UGT1A1 and/or ABCG2 inhibitor is competitive. The regulator compound may also be a SRPK1 inhibitor.

The regulator compound is in some embodiments SCO-101 of the formula:

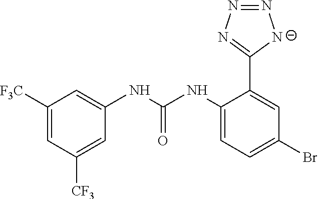 [SMILES: O=C(Nc1cc(C(F)(F)F)cc(C(F)(F)F)c1)Nc1ccc(Br)cc1-c1nnn[n-]1]

or a pharmaceutically acceptable salt thereof. SCO-101 has an IC50 towards UGT1A1 of approximately 0.1 μM.

### Anti-Cancer Agents

The anti-cancer agent(s) of the invention is suitably an UGT1A1 and/or an ABCG2 substrate. In some embodiments the anti-cancer agent(s) is selected from the group consisting of topoisomerase inhibitors, antihormone agents, alkylating agents, mitotic inhibitors, antimetabolites, anti-tumor antibiotics, corticosteroids, targeted anti-cancer therapy agents, differentiating agents, and/or immunotherapy agents, alone or any combination thereof. In particular embodiments the anti-cancer agent(s) is a topoisomerase inhibitor, more particularly a topoisomerase I or topoisomerase II inhibitor. Preferred topoisomerase I inhibitors are those selected from the group consisting of irinotecan, its active metabolite SN-38, and topotecan. In special embodiments the anti-cancer agent is irinotecan and/or its active metabolite SN-38.

A selected (first) anti-cancer agent can further be administered in combination with one or more further anti-cancer agents. Such further anti-cancer agents include 5-fluorouracil, optionally in combination with folinic acid. In a preferred embodiment the anti-cancer agent includes a combination of irinotecan, 5-fluorouracil, and folinic acid (FOLFIRI).

### Treatment and Dosage Regimens

Specific treatment and/or dosage regimes depends on the regulator compound, the anti-cancer agent(s) and the cancer to be treated. In some embodiments, the regulator compound and/or the the anti-cancer agent(s) is administered to the patient daily. Daily administration may be administration once a day or more than once a day, such as twice a day. Daily administration is to be understood as within a 24-hour period of time. The treatment and/or dosage regime may also include treatment cycles comprising periods of treatment interrupted by pauses. Accordingly, a treatment can be repeated a number of times, such as 2 times or more, such as 3 times or more, such as 4 times or more, such as 5 times or more, such as 6 times or more, such as 7 times or more, such as 8 times or more, such as 9 times or more, such as 10 times or more, such as 11 times or more, such as 12 times or more, such as 13 times or more, such as 14 times or more, such as 15 times or more, such as 16 times or more, such as 17 times or more, such as 18 times or more, such as 19 times or more, such as 20 times or more, such as 21 times or more, such as 22 times or more, such as 23 times or more, such as 24 times or more, such as 25 times or more, such as 26 times or more, such as 27 times or more, such as 28 times or more, such as 29 times or more, such as 30 times or more, 31 times or more, such as 32 times or more, such as 33 times or more, such as 34 times or more, such as 35 times or more, such as 36 times or more, such as 37 times or more, such as 38 times or more, such as 39 times or more, such as 40 times or more, such as 41 times or more, such as 42 times or more, such as 43 times or more, such as 44 times or more, such as 45 times or more, such as 46 times or more, such as 47 times or more, such as 48 times or more, such as 49 times or more, such as 50 times or more, such as 51 times or more.

In one embodiment, the treatment regime further comprises administering to the patient a Granulocyte colony-stimulating factor (G-CSF). The administration of the Granulocyte colony-stimulating factor (G-CSF) may be done subsequently to the administration of the anti-cancer agent(s) within a treatment cycle.

Due to the of increase in plasma drug exposure (AUC) and/or the Cₘₐₓ and/or the t_{1/2} of the anti-cancer agent(s) in some embodiments the anti-cancer agent can be administered below the recommended dose according to the Summary of Product Characteristics, while still provide effective treatment at a tolerable toxicity level. In particular the anti-cancer agent(s) dosage can be from 40% to 80% of the Summary of Product Characteristics, such as from 40% to 60% such as from 40 to 41%, such as from 41% to 42%, such as from 42% to 43%, such as from 43% to 44%, such as from 44% to 45%, such as from 45% to 46%, such as from 46% to 47%, such as from 47% to 48%, such as from 48% to 49%, such as from 49% to 50%, such as from 50% to 51%, such as from 51% to 52%, such as from 52% to 53%, such as from 53% to 54%, such as from 54% to 55%, such as from 55% to 56%, such as from 56% to 57%, such as from 57% to 58%, such as from 58% to 59%, such as from 59% to 60%.

In the embodiment where the regulator compound is SCO-101 or a pharmaceutically acceptable salt thereof and the anti-cancer agent comprise irinotecan and/or its active metabolite SN-38, the SCO-101 is advantageously administered in amounts/doses which are toxicologically acceptable, yet sufficient for maintaining a plasma concentration of SCO-101 of at least 10 μg/mL for a period of time of at least 24 hours after administration of the irinotecan or the SN-38. In the embodiment where the regulator compound is SCO-101 or a pharmaceutically acceptable salt thereof and the anti-cancer agent comprises irinotecan and/or its active metabolite SN-38, the SCO-101 is advantageously administered in amounts/doses which are toxicologically acceptable, yet sufficient for maintaining a plasma concentration of SCO-101 of at least 5 μg/mL for a period of time of at least 24 hours after administration of the irinotecan or the SN-38.

In the embodiment where the regulator compound is SCO-101 or a pharmaceutically acceptable salt thereof and the anti-cancer agent comprises irinotecan and/or its active metabolite SN-38, the SCO-101 is advantageously administered in amounts/doses providing an area under the curve (AUC) of SN-38 of more than 385 h*ng/ml from single dose administration of irinotecan or SN-38, such as at least 400 h*ng/ml, such as at least 450 h*ng/ml, such as at least 500 h*ng/ml, such as at least 550 h*ng/ml, such as at least 600 h*ng/ml, such as at least 650 h*ng/ml, such as at least 700 h*ng/ml, such as at least 750 h*ng/ml, such as at least 800 h*ng/ml, such as at least 850 h*ng/ml, such as at least 900 h*ng/ml, such as at least 950 h*ng/ml, such as at least 1000 h*ng/ml, such as at least 1050 h*ng/ml, such as at least 1100 h*ng/ml, such as at least 1150 h*ng/ml, such as at least 1200 h*ng/ml, such as at least 1250 h*ng/ml, such as at least 1300 h*ng/ml, such as at least 1350 h*ng/ml, such as at least 1400 h*ng/ml, such as at least 1450 h*ng/ml, such as at least 1500 h*ng/ml, such as at least 1550 h*ng/ml, such as at least 1600 h*ng/ml, such as at least 1650 h*ng/ml, such as at least 1700 h*ng/ml, such as at least 1750 h*ng/ml, such as at least 1800 h*ng/ml, such as at least 1850 h*ng/ml, such as at least 1900 h*ng/ml, such as at least 1950 h*ng/ml, such as at least 2000 h*ng/ml.

In one embodiment, the regulator compound is SCO-101 or a pharmaceutically acceptable salt thereof, the administered anti-cancer agent comprises irinotecan and SCO-101 is administered in amounts/doses providing an area under the curve (AUC) of SN-38 of from 390 to 3500 h*ng/ml from single dose administration of irinotecan or SN-38, such as from 400 to 3300 h*ng/ml, such as from 450 to 3100 h*ng/ml, such as from 500 to 2900 h*ng/ml, such as from 550 to 2700 550 h*ng/ml, such as from 600 to 2500 h*ng/ml. In some embodiments, SCO-101 or a pharmaceutically acceptable salt thereof is administered in amounts/doses providing an AUC of SN-38 of 6000 h*ng/m or less.

In the embodiment where the regulator compound is SCO-101 or a pharmaceutically acceptable salt thereof and the anti-cancer agent comprises from 25 mg/m2 to 180 mg/m2, such as from 25 mg/m2 to 170 mg/m2 irinotecan and/or its active metabolite SN-38, the SCO-101 is advantageously administered in amounts/doses providing an area under the curve (AUC) of SN-38 of more than 385 h*ng/ml from single dose administration of irinotecan or SN-38, such as at least 400 h*ng/ml, such as at least 450 h*ng/ml, such as at least 500 h*ng/ml, such as at least 550 h*ng/ml, such as at least 600 h*ng/ml, such as at least 650 h*ng/ml, such as at least 700 h*ng/ml, such as at least 750 h*ng/ml, such as at least 800 h*ng/ml, such as at least 850 h*ng/ml, such as at least 900 h*ng/ml, such as at least 950 h*ng/ml, such as at least 1000 h*ng/ml, such as at least 1050 h*ng/ml, such as at least 1100 h*ng/ml, such as at least 1150 h*ng/ml, such as at least 1200 h*ng/ml, such as at least 1250 h*ng/ml, such as at least 1300 h*ng/ml, such as at least 1350 h*ng/ml, such as at least 1400 h*ng/ml, such as at least 1450 h*ng/ml, such as at least 1500 h*ng/ml, such as at least 1550 h*ng/ml, such as at least 1600 h*ng/ml, such as at least 1650 h*ng/ml, such as at least 1700 h*ng/ml, such as at least 1750 h*ng/ml, such as at least 1800 h*ng/ml, such as at least 1850 h*ng/ml, such as at least 1900 h*ng/ml, such as at least 1950 h*ng/ml, such as at least 2000 h*ng/ml.

In the embodiment where the regulator compound is SCO-101 or a pharmaceutically acceptable salt thereof and the anti-cancer agent comprises irinotecan and/or its active metabolite SN-38, the SCO-101 is advantageously administered in amounts/doses maintaining a plasma concentration of SCO-101 of at least 5 μg/ml for a period of time of at least 24 hours after administration of the irinotecan or the SN-38. In other embodiments SCO-101 is administered in amounts/doses which are toxicologically acceptable, yet sufficient for maintaining a plasma concentration of SCO-101 of at least 15 μg/mL, such as at least 20 μg/mL, such as at least 25 μg/mL, such as at least 30 μg/mL, such as 35 μg/mL, such as 40 μg/mL, such as at least 45 μg/mL, such as at least 50 μg/mL, such as at least 60 μg/mL, such as at least 65 μg/mL, such as at least 70 μg/mL, such as at least 75 μg/mL, such as at least 80 μg/mL, such as at least 85 μg/mL, such as at least 90 μg/mL, such as at least 95 μg/mL, such as at least 100 μg/mL for a period of time of at least 24 hours after administration of the irinotecan or the SN-38. Generally, the plasma concentration of SCO-101 should be kept below 200 μg/mL, such as between 30 to 70 μg/mL. In one embodiment, the plasma concentration of SCO-101 should be kept below 200 μg/mL, such as between 10 to 40 μg/mL. In more particular embodiments the SCO-101 or the pharmaceutically acceptable salt thereof is administered in a total daily dose of at least 20 mg, such as at least 30 mg, such as at least 40 mg, such as at least 50 mg, such as at least 60 mg, such as at least 70 mg, such as at least 80 mg, such as at least 90 mg, such as at least 100 mg, such as at least 120 mg, such as at least 140 mg, such as at least 160 mg, such as at least 180 mg, such as at least 200 mg, such as at least 250 mg, such as at least 300 mg, such as at least 350 mg. Generally, the daily dose of SCO-101 should be kept below 400 mg. Preferably, the total daily dose of SCO-101 is 150 mg. The total daily dose of SCO-101 can be administered as a once-daily dose or more preferable in twice-daily doses or even divided into further doses during a 24-hour period to minimize fluctuations on SCO-101 plasma levels.

In the embodiment where the regulator compound is SCO-101 or a pharmaceutically acceptable salt thereof and the anti-cancer agent comprise irinotecan and/or its active metabolite SN-38, the irinotecan and/or SN-38 is advantageously administered in amounts/doses which are toxicologically acceptable, yet sufficient for maintaining a plasma concentration of SN-38 of at least 2 ng/ml for at least 48 hours. In some embodiments where the regulator compound is SCO-101 or a pharmaceutically acceptable salt thereof and the anti-cancer agent comprises irinotecan and/or its active metabolite SN-38, the irinotecan and/or SN-38 is advantageously administered in amounts/doses which are toxicologically acceptable, yet sufficient for maintaining a plasma concentration of SN-38 of at least 1 ng/ml for at least 48 hours. In particular such dose(s) provides for a maximum plasma concentration (Cₘₐₓ) of irinotecan and/or SN-38 of at least 5 ng/ml, such as at least 10 ng/ml, such as at least 15 ng/ml, such as at least 20 ng/ml, such as at least 25 ng/ml, such as at least 30 ng/ml, such as at least 35 ng/ml, such as at least 40 ng/ml, such as at least 45 ng/ml, such as at least 50 ng/ml, such as at least 55 ng/ml, such as at least 60 ng/ml, such as at least 65 ng/ml, such as at least 70 ng/ml, such as at least 75 ng/ml, such as at least 80 ng/ml, such as at least 85 ng/ml, such as at least 90 ng/ml, such as at least 95 ng/ml, such as at least 100 ng/ml.

In one embodiment, SCO-101 or a pharmaceutically acceptable salt thereof is administered in amounts/doses providing a plasma concentration of SN-38 of from 1 ng/ml to 40 ng/ml for at least 48 hours, such as from 1 ng/ml to 10 ng/ml, such as from 10 ng/ml to 15 ng/ml, such as from 15 ng/ml to 20 ng/ml, such as from 20 ng/ml to 25 ng/ml, such as from 25 ng/ml to 30 ng/ml, such as from 30 ng/ml to 35 ng/ml, such as from 35 ng/ml to 40 ng/ml.

In one embodiment, SCO-101 or a pharmaceutically acceptable salt thereof is administered in amounts/doses providing a maximum plasma concentration (Cₘₐₓ) of SN-38 of from 5 ng/ml to 140 ng/ml, such as from 5 ng/ml to 10 ng/ml, such as from 10 ng/ml to 15 ng/ml, such as from 15 ng/ml to 20 ng/ml, such as from 20 ng/ml to 25 ng/ml, such as from 25 ng/ml to 30 ng/ml, such as from 30 ng/ml to 35 ng/ml, such as from 35 ng/ml to 40 ng/ml, such as from 40 ng/ml to 45 ng/ml, such as from 45 ng/ml to 50 ng/ml, such as from 50 ng/ml to 55 ng/ml, such as from 55 ng/ml to 60 ng/ml, such as from 60 ng/ml to 65 ng/ml, such as from 65 ng/ml to 70 ng/ml, such as from 70 ng/ml to 75 ng/ml, such as from 75 ng/ml to 80 ng/ml, such as from 80 ng/ml to 85 ng/ml, such as from 85 ng/ml to 90 ng/ml, such as from 90 ng/ml to 95 ng/ml, such as from 95 ng/ml to 100 ng/ml, such as from 100 ng/ml to 105 ng/ml, such as from 105 ng/ml to 110 ng/ml, such as from 110 ng/ml to 115 ng/ml, such as from 115 ng/ml to 120 ng/ml, such as from 120 ng/ml to 125 ng/ml, such as from 125 ng/ml to 130 ng/ml, such as from 130 ng/ml to 135 ng/ml, such as from 135 ng/ml to 140 ng/ml.

Generally, the plasma concentration of SN-38 should be kept below 150 ng/ml. In some embodiments, irinotecan is administered in amounts from 20% to 95% of the recommended dose according to the Summary of Product Characteristics, such as from 20% to 25%, such as from 25% to 30%, such as from 30% to 35%, such as from 35% to 40%, such as from 40% to 45%, such as from 45% to 50%, such as from 50% to 55%, such as from 55% to 60%, such as from 60% to 65%, such as from 65% to 70%, such as from 70% to 75%, such as from 75% to 80%, such as from 80% to 85%, such as from 85% to 90%, such as from 90% to 95%. In further embodiments the irinotecan and/or SN-38 is administered in a total daily dose which is at most 40% to 80% of the recommended dose according to the Summary of Product Characteristics, such as at most 40% to 60% such as from 40 to 41%, such as from 41% to 42%, such as from 42% to 43%, such as from 43% to 44%, such as from 44% to 45%, such as from 45% to 46%, such as from 46% to 47%, such as from 47% to 48%, such as from 48% to 49%, such as from 49% to 50%, such as from 50% to 51%, such as from 51% to 52%, such as from 52% to 53%, such as from 53% to 54%, such as from 54% to 55%, such as from 55% to 56%, such as from 56% to 57%, such as from 57% to 58%, such as from 58% to 59%, such as from 59% to 60%, such as from 60% to 61%, such as from 61% to 62%, such as from 62% to 63%, such as from 63% to 64%, such as from 64% to 65%, such as from 65% to 66%, such as from 66% to 67%, such as from 67% to 68%, such as from 68% to 69%, such as from 69% to 70%, such as from 70% to 71%, such as from 71% to 72%, such as from 72% to 73%, such as from 73% to 74%, such as from 74% to 75%, such as from 75% to 76%, such as from 76% to 77%, such as from 77% to 78%, such as from 78% to 79%, such as from 79% to 80% of 180 mg/m2. More particularly, the irinotecan and/or SN-38 is administered in a total daily dose of less than 110 mg/m2. More particularly, the irinotecan and/or SN-38 is administered in a total daily dose of 110 mg/m2 or less. In some embodiments, the irinotecan and/or SN-38 is administered in a total daily dose of 120 mg/m2 or less. In some embodiments, the irinotecan and/or SN-38 is administered in a total daily dose of 130 mg/m2 or less. In some embodiments, the irinotecan and/or SN-38 is administered in a total daily dose of 140 mg/m2 or less. In some embodiments, the irinotecan and/or SN-38 is administered in a total daily dose of 150 mg/m2 or less. In some embodiments, the irinotecan and/or SN-38 is administered in a total daily dose of 160 mg/m2 or less. In some embodiments, the irinotecan and/or SN-38 is administered in a total daily dose of 170 mg/m2 or less. In some embodiments, the irinotecan and/or SN-38 is administered in a total daily dose of 180 mg/m2 or less. Similar to dosing SCO-101, the total daily dose of irinotecan and/or SN-38 can be administered as a once-daily dose or as twice-daily doses of a lesser amount or even divided into further doses during a 24-hour period to minimize fluctuations in irinotecan and/or SN-38 plasma levels.

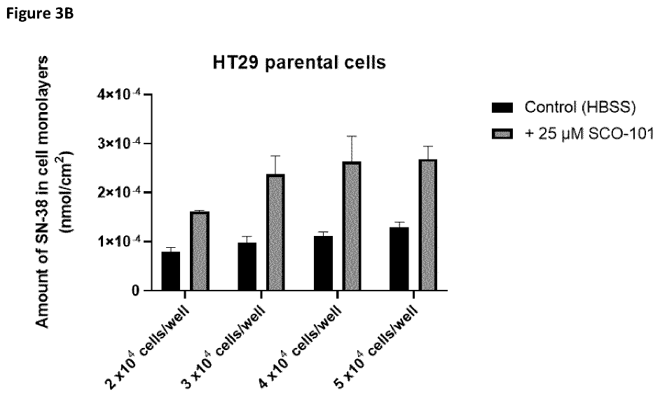

In some embodiments, SCO-101 or a pharmaceutically acceptable salt thereof is administered in a total daily dose of from 20 mg to 400 mg, such as from 50 mg to 350 mg, such as from 100 mg to 300 mg; and the anti-cancer agent, such as irinotecan, is administered in a total daily dose of 110 mg/m2 or less, such as 100 mg/m2 or less, such as 90 mg/m2 or less, such as 80 mg/m2 or less, such as 70 mg/m2 or less, such as 60 mg/m2 or less. Based on the significantly increased up concentration of SN-38 by SCO-101 in both resistant and parental cancer cells, shown in FIG. 3A-D, the dose of the anti-cancer agent, such as irinotecan can be lowered while maintaining at least similar or superior therapeutic anti-cancer effect.

Amounts with respect to the regulator compound, such as SCO-101 are based on the non-salt form of the compound, for example the free acid of SCO-101.

### Cancers

Cancers, which can advantageously be treated by the method of the invention, include but is not limited to, cancers which forms solid tumors, such as sarcomas, carcinomas and lymphomas. In some embodiments the cancers is or know to become a metastatic cancer.

In some embodiments the cancer is selected from the group consisting of colorectal cancer, breast cancer, lung cancer (including non small cell lung cancer and small cell lung cancer), glioblastomas, head and neck cancers, malignant melanomas, basal cell skin cancer, squamous cell skin cancer, liver cancer, pancreatic cancer, prostate cancer, anal cancer, cervix uteri cancer, bladder cancer, corpus uteri cancer, ovarian cancer, gall bladder cancer, leukemia's (including myeloid and lymphatic leukemia), and/or myelomatosis. In further embodiments the cancer includes metastatic colorectal cancer; metastatic pancreatic cancer and/or metastatic breast cancer.

In particular embodiments the cancer to be treated is or becomes resistant to the anti-cancer agent when administered without the regulator compound of the invention. Such resistance may be de novo resistance and/or it may be an acquired resistance. In some embodiments the regulator compound re-sensitizes the cancer, especially resistant cancer to the anti-cancer agent.

### Combination Drugs

In a further aspect the invention provides a combination drug comprising, separately or together,
a) an anti-cancer agent, and
b) a regulator compound,
wherein the amount of the regulator compound effectively increases the plasma drug exposure (AUC) of the anti-cancer agent when administering the combination drug to a patient.

### EXAMPLES

### Materials and Methods

### Materials

Chemicals used in the examples herein e.g. for buffers and substrates were of pharmaceutically acceptable grade.

### Methods

Patients with metastatic colorectal cancer were included in a study and treated with a combination of SCO-101 and FOLFIRI (combination of irinotecan, 5-FU and leucovorin) in 14 day treatment cycles. Patients received varying doses of Irinotecan between 50% and 80% of the recommended dose of Irinotecan (180 mg/m²) according to their individual tolerability, which is standard practise. On days 1, 2, 3, 4, 5 and 6 in each treatment cycle, patients received one fixed daily dose of 150 mg SCO-101 taken in the morning at least 30 minutes before ingestion of food. On day 5 to 7, patients received FOLFIRI as specified for Colorectal cancer combination regimen 2.

Blood samples were taken from patients at predefined timepoints according to the study protocol. During the first cycle of treatment, blood samples for pharmacokinetic analysis of SCO-101, irinotecan, and SN-38 were taken as follows:
Day 1; (pre-SCO-101 administration, 1; 4; 5; 6; 8); only for PK analysis of SCO-101
Day 2; (24 hours and pre-SCO-101 administration); only for PK analysis of SCO-101
Day 5; (pre-SCO-101 administration, 1; 4; 5; 6; 8)
Day 6; (24 hours and pre-SCO-101 administration),
Day 7; (48 hours),
Day 9; (96 hours).

Blood samples were collected in 4 mL blood-collection tubes using lithium heparin as the anti-coagulant. Blood samples were centrifuged, and the resulting plasma is withdrawn and stored at −70° C. until analysis.

Blood plasma samples were analysed for concentrations of SCO-101, irinotecan and SN-38 using Liquid Chromatography with Tandem Mass Spectrometric Detection (LC-MS/MS) bioanalytical method calibrated and validated in the rant 300 to 90.000 ng/ml. The method and system, including validation were according to industry standard. Data were analysed using commercially available software such as Win-Non-Lin (available from Certara inc.)

### Example 1—Plasma Concentrations of SCO-101 and SN-38 in a Human Patient Upon Repeated Administration of SCO-101

SCO-101 and Irinotecan was administered as described in the materials and methods section. Blood samples were withdrawn and analysed as specified. Blood samples taken on day 1 and day 2 were only analysed for SCO-101 while blood samples taken on days 5, 6, 7 and 9 were analysed for SCO-101, Irinotecan and SN-38. Raw data was then plotted using win-non-lin software to obtain relevant pharmacokinetic parameters, e.g. Cₘₐₓ and AUC.

Based on the data obtained, the pharmacokinetic profile for SCO-101 was modelled to show the increase in plasma concentrations of SCO-101 over the full 6 days. No data is available for the exposure profile of SCO-101 between 24 hours after the first SCO-101 dose and until 96 hours (day 5), however the model is considered an accurate representation of the data as it gives a good fit to the observed data on day 1, day 2, day 5, day 6, day 7 and day 9.

As patients received varying doses of Irinotecan, the pharmacokinetic profile of SN-38 was prepared for each individual patient and subsequently normalised to the same dose to enable comparison and modelling of the data. This is common standard practice in the art for evaluating pharmacokinetic data where different doses are administered.

Reference data regarding pharmacokinetic parameters for known standard irinotecan formulation and pegylated liposomal irinotecan, as available under the registered trademark Onivyde® was retrieved from publicly sources. Pharmacokinetic data for the standard irinotecan formulation is obtained from publically available databases and data from the pegylated liposomal formulation was obtained from the EMA public assessment report (EPAR) on Onivyde® pegylated liposomal (https://www.ema.europa.eu/en/documents/assessment-report/onivyde-epar-public-assessment-report_en.pdf).

### Conclusion

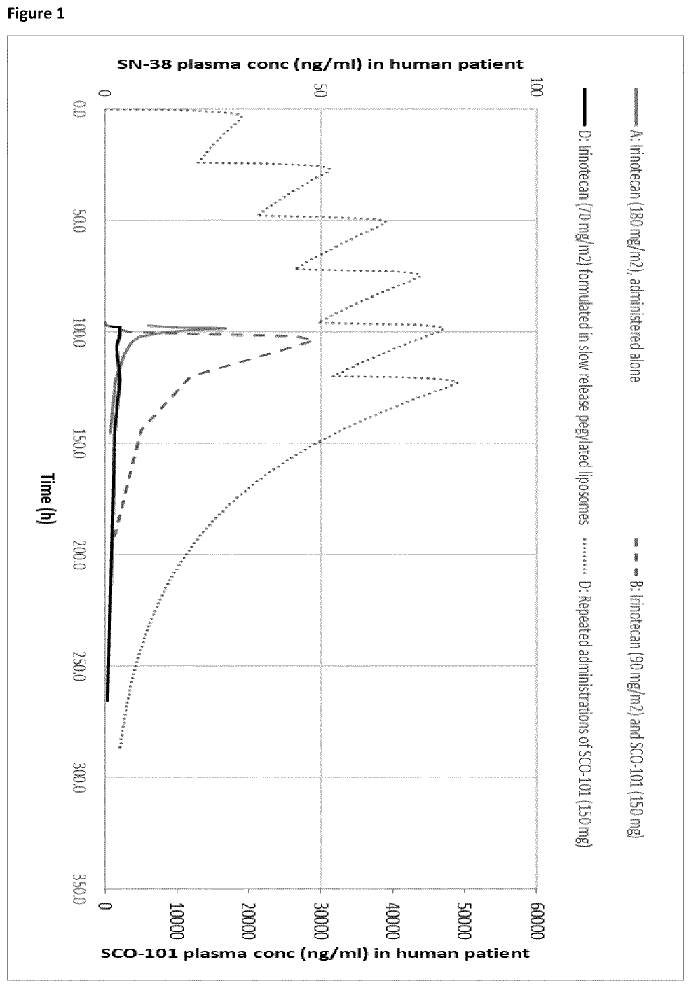

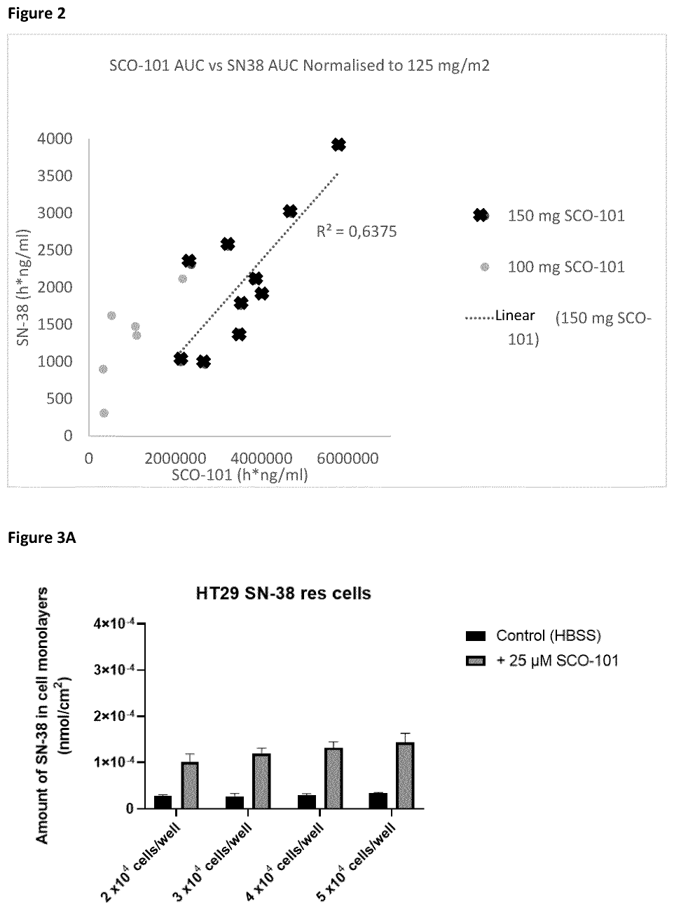

The resulting pharmacokinetic data are shown in FIG. 1.

FIG. 1D shows the plasma concentrations of SCO-101 when administered in doses of 150 mg repeated every 24 hours. It is observed that for this particular regime the plasma levels over time appear to stabilize around 55 μg/mL, while wearing off with a half-life of about 24 hours after stopping the administration.

FIG. 1B shows that when also administering SCO-101, irinotecan given in a dose (90 mg/m²) only half the recommended dose according to the Summary of Product Characteristics (180 mg/m²), still results in a Cₘₐₓ which is almost double the Cₘₐₓ in conventional treatment, while the plasma drug exposure (AUC) increased many folds.

FIG. 1A shows that administering irinotecan in a conventional regime according to the Summary of Product Characteristics (180 mg/m²), results in a steep rise in SN-38 plasma concentration generating a Cₘₐₓ of about 25 ng/ml, and an almost equally steep decline in SN-38 plasma concentration, which becomes therapeutically ineffective after only about 24 hours.

FIG. 1C illustrates, that when administering irinotecan formulated in a known slow release pegylated liposome (70 mg/m²) hardly any peak Cₘₐₓ is observed, rather the formulation results in a low plasma concentration of SN-38 which, although lasting longer than in the conventional treatment, only provides for a limited therapeutic effect.

In conclusion these data clearly show a superior plasma drug exposure (AUC), Cₘₐₓ and t_{1/2}, when administering irinotecan together with SCO-101 in the selected dosage regime.

### Example 2—Evaluation of SN-38 PK Profile in Patients with Metastatic Colorectal Cancer Treated with a Combination of SCO-101 and FOLFIRI

A trial was setup to address the safety, tolerability, and efficacy of SCO-101 given orally for 6 days followed by FOLFIRI at varying doses from day 5 to 7, in a biweekly schedule, in patients with metastatic colorectal cancer who have formerly been treated with FOLFIRI and afterwards progressed. The first part of the study was a dose-finding study, where the impact of SCO-101 on the pharmacokinetics (PK) of SN-38 was studied.

### Study Population:

12 patients from the dose-finding part of the study received 150 mg SCO-101 for 6 days and 45-80% of the recommended dose of FOLFIRI. 6 of the patients had RAS wildtype tumors and these patients were subjected to further analysis.

### Method:

Blood for pharmacokinetic analysis was sampled from the patients were taken at 1, 2, 4, 8, 24, 48, and 96 hours after treatment with FOLFIRI and SCO-101. The blood samples were analyzed for Cₘₐₓ, T_{1/2} and AUC (0-24 h) of SCO-101, irinotecan and SN-38.

### Results:

The pharmacokinetics of SN-38 from the patients in the study, normalized to a dose of irinotecan of 90 mg/m2 showed a T_{1/2} on day 5 of 19 hours (SD 5·7) a Cₘₐₓ of 60 ng/ml (SD 20.6) and an AUC_0-24 h of 1415 h*ng/ml (SD: 670).

The results were compared to SN-38 data from standard treatment with irinotecan at 180 mg/m2, which is used in standard doses of FOLFIRI and the data is presented in Table 1.

TABLE 1
Comparison of SN-38 PK data between standard irinotecan and data from study

| | Dose | | | | AUC_0-24 |
|---|---|---|---|---|---|
| | irinotecan/m2 | T½ hours (SD) | | Cmax ng/ml (SD) | h*ng/ml (SD) |
| SN-38 Standard | 180 mg | 11.7 | (4.3) | 40 (11.6) | 385 (115) |
| SN-38 study | 90 mg | 19 | (5.7) | 60 (20.6) | 1415 (670) |

| | | | | |
|---|---|---|---|---|
| Fold increase (study | 0.5 | 1.6 | 1.5 | 3.7 |
| vs Standard) | | | | |

The data analysis showed an increased T_{1/2}, increased Cₘₐₓ and increased AUC of SN-38 when combining SCO-101 with 90 mg/m2 irinotecan, compared to SN-38 PK data from standard irinotecan treatment of 180 mg/m2. The toxicity profile of the patients treated with 90 mg irinotecan/m2 (50% FOLFIRI) showed only grade 1 and 2 adverse events. SCO-101 in combination with FOLFIRI has thus demonstrated the ability to modulate the pharmacokinetic profile of SN-38 in metastatic colorectal cancer patients with RAS wildtype tumors, by significantly increasing the half-life, the peak plasma concentration, and area under the curve of SN-38. The combined treatment was well tolerated.

### Example 3—Evaluation of SN-38 Plasma Concentrations in Patients with Metastatic Colorectal Cancer Treated with a Combination of SCO-101 and FOLFIRI

### Study Population:

10 patients received 150 mg SCO-101 orally for 6 days followed by FOLFIRI from day 5 to 7 at 45-80% of the recommended dose. 6 patients received 100 mg SCO-101 orally for 6 days followed by FOLFIRI from day 5 to 7 at 50% of the recommended dose.

### Method:

Blood for pharmacokinetic analysis was sampled from the patients at 1, 2, 4, 8, 24, 48, and 96 hours after treatment with FOLFIRI and SCO-101. The blood samples were analyzed for Cmax, T_{1/2} and AUC (0-24 h) of SCO-101, irinotecan and SN-38.

### Results:

SCO-101 AUC plasma levels correlated with SN-38 AUC plasma levels when normalized to 125 mg/m2 dose as shown in Error! Reference source not found.

### Example 4—Evaluation of SCO-101 Effect on ABCG2 in SN-38-Resistant Human Colon Cancer Cells

Pairs of parental (sensitive) and SN38-resistant human colon cancer cell lines (HT29 and LoVo) were used. ABCG2 overexpression and effects of SCO-101 on ABCG2 was investigated by western blot, qPCR, dye-flux assays, bi-directional flux assays, 3H-SN38 accumulation assays and vesicular uptake assays. The effect of SCO-101 on SN38 re-sensitization was investigated by cell viability assays (MTT/PrestoBlue) and clonogenic assays.

### Example 5—Evaluation of In Vitro UGT1A1 Inhibition by SCO-101

The inhibition of UGT1A1 was investigated by recombinant human enzyme assays.

### Results of Example 4 and 5

Data from the various flux assays demonstrated that SCO-101 inhibited the activity of ABCG2. Protein analysis demonstrated that SCO-101 causes degradation of ABCG2 and in silco docking predicted SCO-101 to bind in the part of ABCG2 where also SN-38 binds. Exposing SN-38-resistant cells to the combination of SCO-101 and SN-38, had a synergistic inhibitory effect on cell viability and colony formation compared to either drug alone.

Furthermore, SCO-101 was demonstrated to be a potent inhibitor of UGT1A1.

### Conclusion from Example 4 and 5

The preclinical studies demonstrated that SCO-101 can re-sensitize SN-38-resistant colon cancer cells to SN-38 through inhibition/degradation of the ABCG2/BCRP drug efflux pump.

Importantly, SCO-101 is also a potent inhibitor of UGT1A1, leading to increased and prolonged exposure of SN-38 in patients receiving irinotecan containing treatment, as observed in an ongoing Phase II clinical trial. Further SCO-101 represents a unique drug compound with a dual mechanism of action.

### Example 6—Evaluation of SCO-101 and Other Kinase Inhibitors on BCRP-Mediated Efflux Transport of SN-38

### Materials and Methods

| | Cat. No. |
|---|---|
| Bovine serum albumin (BSA), Sigma-Aldrich (Brøndby, Denmark) | A7906-100g |
| 10 x Hank's balanced salt solution (HBSS), Gibco, Life technologies (Taastrup, Denmark) | 14065-049 |
| Fetal bovine serum (FBS), Hyclone, GE Healthcare Life Science (Logan, Utah) | SH30088.03 |
| 2-[4-(2-hydroxyethyl) piperazin-1-yl] ethanesulfonic acid (HEPES), Sigma-Aldrich (Brøndby, Denmark) | H7006-100g |
| Triton X-100, AppliChem GmbH (Darmstadt, Germany) | A4975 |
| Ultima Gold Scintillation Fluid, PerkinElmer (Boston, MA) | 6013329 |
| 12-well Transwell ® Permeable supports (1.13 cm², 0.4 μm pore size), Corning, Sigma-Aldrich (Brøndby, Denmark) | CLS3401 |
| 96-well polystyrene flat-bottomed culture plates (0.32 cm²) Corning, Sigma-Aldrich (Brøndby, Denmark) | CLS3595 |
| KO143, Sigma-Aldrich (Brøndby, Denmark) | K2144-5mg |
| Zosuquidar (ZSQ), Selleck Chemicals (Munich, Germany) | LY335979 |
| DMEM (StableCell ™ DMEM - high glucose) Sigma-Aldrich (Brøndby, Denmark) | D0819 |
| P/S (Penicillin-Streptomycin solution, 100x) Sigma-Aldrich (Brøndby, Denmark) | P0781 |
| L-glut (L-Glutamine solution 200 mM) Sigma-Aldrich (Brøndby, Denmark) | G7513 |
| NEAA (MEM Non-essential Amino Acid Solution, 100x) Sigma-Aldrich (Brøndby, Denmark) | M7145 |
| Sodium-Bicarbonate 7.5% solution, Gibco, Life technologies (Taastrup, Denmark) | 25080-060 |
| PBS (Dulbecco's Phosphate Buffered Saline) Sigma-Aldrich (Brøndby, Denmark) | D8537 |
| SCO-101 (Scandion Oncology ) | — |
| SCO-201 (Scandion Oncology) | — |
| Sphinx31 (Scandion Oncology) | — |
| Srpin340 (Scandion Oncology) | — |
| Tomivorsetib (Scandion Oncology) | — |
| CGP57380 (Scandion Oncology) | — |

### Uptake Experiments in Parental HT29 Cell Monolayers and HT29 SN-38 Resistant (SN-38 Res) Cell Monolayers:

For uptake experiments, cells from a HT29 parental and a HT29 SN38-resistant colorectal cancer cell line (SN-38 res) were seeded on the bottom of a 96-well plates (NUNC) generally at a density of 20×103 cells/well and cultured for approximately 48 hours prior to experimentation. However, some wells were used to study the effect of seeding density on the uptake of 3H-SN-38 (1 μCi/mL, 0.07 μM) in the presence and absence of 25 μM SCO-101. In these wells, 2×104, 3×104, 4×104 and 5×104 cells/well were seeded in three technical replicates (N=3). In the remaining wells of the plate setup, the uptake of ³H-SN-38 was investigated in the presence of 1 μM KO143, inhibitor: SCO-101 (100 to 0.5 μM) and four kinase inhibitors (Sphinx31, Srpin340, Tomivorsetib and CGP57380, 25 μM) dissolved in HBSS transport buffer (HBSS, 0.05% BSA, 10 mM HEPES, pH 7.4).

All treatments were investigated in three technical replicates (N=3). On the day of experimentation, the culture media was exchanged with 100 μL transport buffer (HBSS, 0.05% BSA, 10 mM HEPES, pH 7.4) and cells were incubated for 15 minutes at 37° C.

Subsequently, the blank transport buffer was exchanged with 50 μL transport buffer containing the different compounds (in 2x of the final concentration) to be tested and incubated for 15 minutes at 37° C. The uptake experiment was started by adding 50 μL of ³H-SN38 at a concentration of 2 μCi/mL to all wells (the resulting concentration of ³H-SN38 during the experiment was 1 μCi/mL). The uptake experiment was stopped after 15 minutes of incubation at 37° C. by removing the transport buffer from all wells.

The cell monolayers were subsequently washed twice by adding/removing ice-cold transport buffer twice and finally the cell monolayers were lysed by incubation in 100 μl of 0.1% TritonX-100 for 15 minutes at ambient temperature. Cell lysates were transferred to scintillation vials containing 2 mL of scintillation fluid (Ultima Gold, PerkinElmer). Each well was washed with 2×100 μL purified water (milliQ), which was also transferred to respective scintillation vials. The radioactivity in each scintillation vial was measured by means of scintillation counting (Tri-Carb 2910 TR, PerkinElmer, Waltham, MA, USA).

### Results

### Effect of Seeded Cell Density on the Uptake of SN-38 in the Presence or Absence of 25 μM SCO-101

For monolayers of both cell lines and all seeded cell densities, an increased uptake of SN-38 was observed in the presence of 25 μM SCO-101, which indicates inhibition of breast cancer resistance protein-mediated efflux of SN-38. For the SN-38 resistant cell line there only seemed to be a negligible effect on increasing the seeded cell density on the uptake ratio of SN-38 in the presence and absence of 25 μM SCO-101. However, for the HT29 parental cell line, an increase in the ratio between uptake of SN-38 in the presence and absence of 25 μM SCO-101 was observed between seeding densities of 2×10⁴ and 3×10⁴ cells/well (FIG. 1, BOTTOM).

FIG. 3 summarizes the uptake of SN-38 in cell monolayers of both the SN-38 resistant (FIG. 3A) and the parental (FIG. 3B) HT29 cell lines.

### Concentration Dependent Effect of SCO-101 on the Uptake of SN-38 in Monolayers of the HT29 SN-38 Resistant and Parental Cell Lines

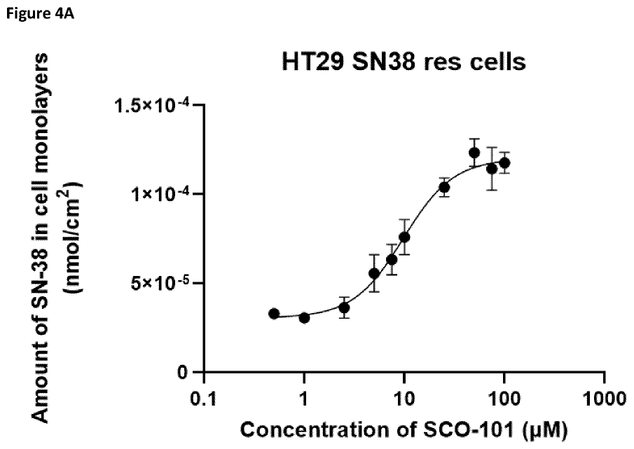

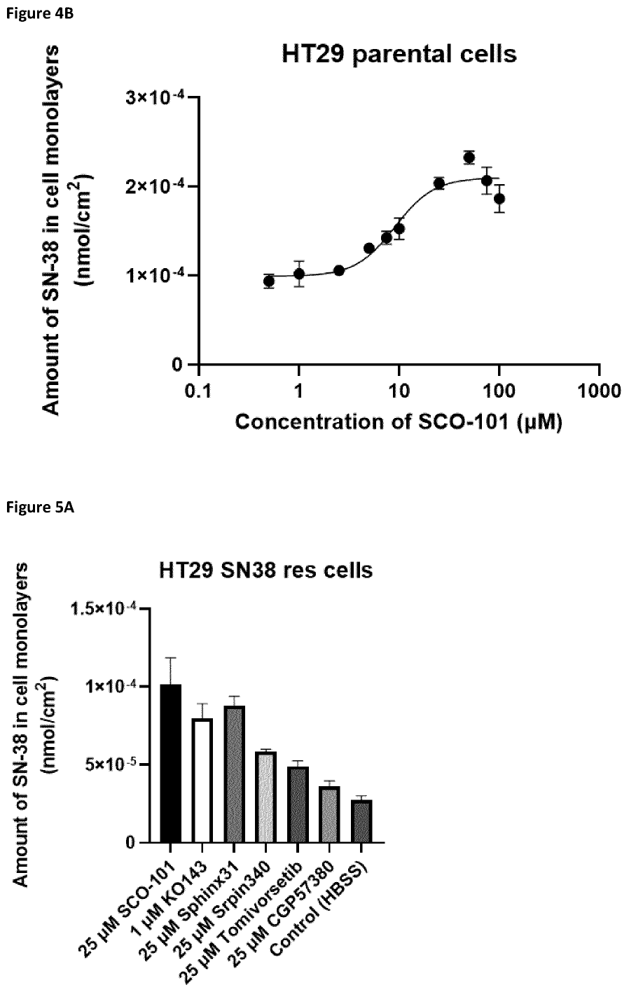

In monolayers of both cell lines, a concentration-dependent increase in uptake of SN-38 was observed (FIG. 4). In monolayers of the HT29 SN-38 res cells, the estimated EC50-values was 9.8 μM, while the estimated EC50-value for SCO-101 was 9.1 in monolayers of the HT29 parental cell line.

FIG. 4 summarizes the effect of increasing concentrations of SCO-101 on the uptake of SN-38 in monolayers of the HT29 SN-38 resistant and parental cell lines.

### Effect of Kinase Inhibitors on the Uptake of SN-38 in Monolayers of the HT29 SN-38 Resistant and Parental Cell Lines

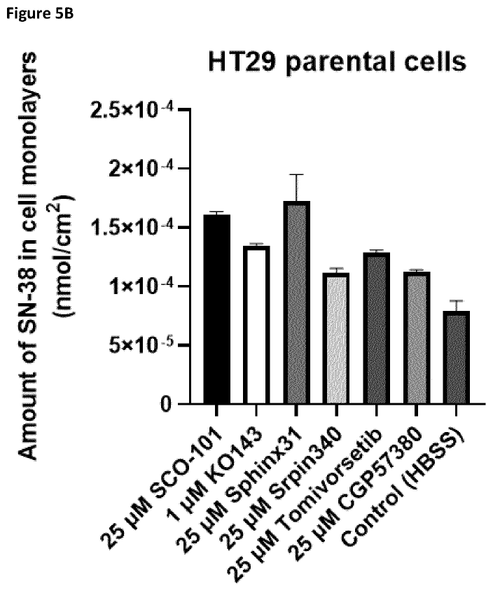

FIG. 5 summarizes the effect of four kinase inhibitors (Sphinx31, Srpin340, Tomivorsetib and CGP57380), 25 μM SCO-101 and 1 μM KO143 on the uptake of SN-38 in monolayers of of the HT29 SN-38 resistant and parental cell lines.

In monolayers of both cell lines, a marked increase in SN-38 uptake was observed in the presence of 25 μM of Sphinx31 relative to the SN-38 uptake in control wells. The SN-38 uptake in the presence of this compound was comparable to the uptake in the presence of 1 μM KO143 and 25 μM SCO-101 (FIG. 6). In monolayers of the SN-38 resistant cells, the uptake of SN-38 was significantly increased in the presence of Sphinx31, Srpin340 and Tomivorsetib of at a concentration of 25 μM, and also in the presence of 1 μM KO143 and 25 μM SCO-101, relative to the uptake in monolayers exposed to blank HBSS transport buffer (ANOVA, α=0.05). However, the uptake of SN-38 was not different in the presence of 25 μM CGP5738. In the HT29 parental cell line, the uptake of SN-38 was significantly increased in the presence of all four kinase inhibitors at a concentration of 25 μM, 1 μM KO143 and 25 μM SCO-101, relative to the uptake in monolayers exposed to blank HBSS transport buffer (ANOVA, α=0.05).

A concentration-dependent increase in SN-38 was observed for SCO-101 in both the HT29 parental and the SN-38 resistant cell lines. Of the four tested kinase inhibitors, a marked increase in SN-38 was observed for Sphinx31 at the tested concentration of 25 μM in both cell lines. Srpin340 and Tomivorsetib caused a significant increase in the uptake of SN-38 at a concentration of 25 μM. The fourth kinase inhibitor, CGP57380 only caused a significant increase in SN-38 uptake in the HT29 parental cell line, but not in the SN-38 resistant cell line.

### Conclusion

The present example demonstrates that SCO-101 can effectively and favourably modulate the pharmacokinetic properties of an anti-cancer agent, such as SN-38 in both resistant and parental cancer cells. Hence, regulator compounds such as SCO-101 are promising candidates for potentiating the therapeutic effect of anti-cancer agents, allowing for administration of anti-cancer agents at reduced doses, improving the anti-cancer agents' risk-benefit profile and overcoming cancer resistance.

### Example 7—Evaluation of SN-38 PK Profile in Patients with Metastatic Colorectal Cancer Treated with a Combination of SCO-101 and FOLFIRI

### Materials and Methods

This example was conducted using essentially similar materials and methods as in Example 2.

### Results

TABLE 2
Comparison of SN-38 PK data between standard irinotecan and data from study

| | | | | | | | *Literature | |
|---|---|---|---|---|---|---|---|---|
| | | | | | | | value | |
| | | | Irinotecan | | Irinotecan | | Irinotecan | |
| | | | (FOLFIRI) | | (FOLFIRI) | | (Camptosar) | |
| | | | Norm 125 | | Norm 125 | | Norm 125 | |
| | | | mg/m2 | | mg/m2 | | mg/m2 | |
| | PK | | (n = 6) | | (n = 10) | | (n = 99) | |
| Analyte | parameters | Unit | Mean (SD) | | Mean (SD) | | Mean (SD) | |
| Add-On | | | 100 mg | | 150 mg | | NO | |
| | | | SCO-101 | | SCO-101 | | SCO-101 | |
| Irinotecan | AUC | h · ng/ml | 10723 | (3306) | 12565 | (5097) | 10529 | (37869) |
| | Cmax | ng/ml | 1348 | (355) | 1457 | (334) | 1492 | (452) |
| | Clearance (CL) | L/h/m2 | 13 | (4) | 14 | (15) | 13.0 | (5.6) |
| SN-38 | AUC | h · ng/ml | 1297 | (624) | 2088 | (911) | 267 | (115) |
| | Cmax | ng/ml | 80 | (40) | 86 | (27) | 27.8 | (11.6) |

### Conclusions

This demonstrate that SCO-101 significantly enhance the AUC and Cmax of SN-38 compared to irinotecan when used in monotherapy. 100 mg SCO-101 provided an AUC of 1297 h*ng/ml compared to 267 h*ng/ml for 125 mg/m2 irinotecan administered alone as single dose, whereas 150 mg SCO-101 provided an AUC of 2088 h*ng/ml compared to 267 h*ng/ml for 125 mg/m2 irinotecan administered alone as single dose, corresponding to an approximately 5-fold and 8-fold increase in AUC for 100 mg SCO-101 and 150 mg SCO-101, respectively.

1-45. (canceled)

46. A method for treatment of cancer in a patient comprising administering a combination drug to the patient comprising, separately or together:
a) an anti-cancer agent; and
b) a regulator compound, wherein an amount of the regulator compound effectively increases the plasma drug exposure (AUC) of the anti-cancer agent when administering the combination drug to a patient.

47. The method of claim 46, wherein the regulator compound is an inhibitor of the degradation, clearance, or binding of the anti-cancer agent or a therapeutically active metabolite thereof

48. The method of claim 46, wherein the plasma drug exposure (AUC) or the plasma half-life (t½) is increased by more than 25% compared to administering the anti-cancer agent without administering the regulator compound.

49. The method of claim 46, wherein the administration of the anti-cancer agent provides for a maximum plasma concentration (Cmax) of the anti-cancer agent and wherein the Cmax is increased compared to the Cmax when administering the anti-cancer agent without the regulator compound.

50. The method of claim 46, wherein the plasma drug exposure (AUC) of the administered anti-cancer agent is higher than the plasma drug exposure (AUC) of the anti-cancer agent administered at a higher dose, but without administering the regulator compound.

51. The method of claim 46, wherein the regulator compound is an UGT1A1 or ABCG2 inhibitor.

52. The method of claim 46, wherein the regulator compound is SCO-101 of the formula: 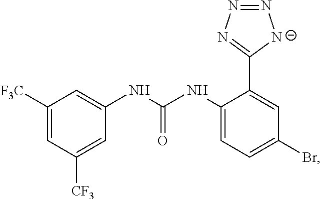 [SMILES: O=C(Nc1cc(C(F)(F)F)cc(C(F)(F)F)c1)Nc1ccc(Br)cc1-c1nnn[n-]1]
or a pharmaceutically acceptable salt thereof.

53. The method of claim 46, wherein, the anti-cancer agent is an UGT1A1 or an ABCG2 substrate.

54. The method of claim 46, wherein the anti-cancer agent is selected from the group consisting of a topoisomerase inhibitor, an antihormone agent, an alkylating agent, a mitotic inhibitor, an antimetabolite, an anti-tumor antibiotic, a corticosteroid, a targeted anti-cancer therapy, a differentiating agent, and an immunotherapy.

55. The method of claim 54, wherein the anti-cancer agent is a topoisomerase I inhibitor selected from the group consisting of irinotecan, its active metabolite SN-38, and topotecan.

56. The method of claim 46, wherein the regulator compound is SCO-101 or a pharmaceutically acceptable salt thereof, the administered anti-cancer agent comprises irinotecan and wherein the SCO-101 is administered in amounts or doses providing an area under the curve (AUC) of SN-38 of more than 385 h*ng/ml from single dose administration of irinotecan or SN-38.

57. The method of claim 46, wherein the regulator compound is SCO-101 or a pharmaceutically acceptable salt thereof, the administered anti-cancer agent comprises irinotecan and wherein the SCO-101 is administered in amounts or doses providing an area under the curve (AUC) of SN-38 of from 390 to 3500 h*ng/ml from single dose administration of irinotecan or SN-38.

58. The method of claim 46, wherein the regulator compound is SCO-101 or a pharmaceutically acceptable salt thereof, the administered anti-cancer agent comprises from 25 mg/m² to 170 mg/m² irinotecan and wherein the SCO-101 is administered in amounts or doses providing an area under the curve (AUC) of SN-38 of more than 385 h*ng/ml from single dose administration of irinotecan or SN-38.

59. The method of claim 46, wherein the regulator compound is SCO-101 or a pharmaceutically acceptable salt thereof administered in a total daily dose of from 20 mg to 400 mg.

60. The method of claim 46, wherein the anti-cancer agent is irinotecan and is administered in toxicologically acceptable doses maintaining a plasma concentration of its active metabolite SN-38 of at least 1 ng/ml for at least 48 hours.

61. The method of claim 55, wherein irinotecan is administered in:
a) amounts from 20% to 95% of the recommended dose according to the Summary of Product Characteristics; or
b) a total daily dose of of 170 mg/m² or less.

62. The method of claim 46, wherein the cancer is selected from the group consisting of colorectal cancer, breast cancer, lung cancer, glioblastomas, head and neck cancers, malignant melanomas, basal cell skin cancer, squamous cell skin cancer, liver cancer, pancreatic cancer, prostate cancer, anal cancer, cervix uteri cancer, bladder cancer, corpus uteri cancer, ovarian cancer, gall bladder cancer, leukemia's, and myelomatosis.

63. The method of claim 46, wherein the cancer is:
a) metastatic colorectal cancer;
b) metastatic pancreatic cancer,
c) metastatic breast cancer.

64. The method of claim 46, wherein the cancer is a resistant cancer which is resistant to the anti-cancer agent when administered alone.

65. A method for increasing the plasma drug exposure (AUC) of an anti-cancer agent in a patient receiving the anti-cancer agent for treatment of a cancer, the method comprising administering a regulator compound to the patient, wherein the regulator compound is an inhibitor of the degradation, clearance, or binding of the anti-cancer agent or a therapeutically active metabolite thereof, thereby increasing the plasma drug exposure of the anti-cancer agent.
